(19) United States
(12) Patent Application Publication
(10) Pub. No.: US 2017/0055901 A1
(43) Pub. Date: Mar. 2, 2017

(54) THERAPEUTIC AGENT DELIVERING BIOPSY NEEDLE DEVICE AND METHOD OF USE

(71) Applicant: ALABASTER TECHNOLOGIES, INC., Vancouver (CA)

(72) Inventors: Colin NYULI, Vancouver (CA); Dennis Lyle JANZEN, Vancouver (CA); Douglas Glen JANZEN, Vancouver (CA); Randy Matthew LANE, Langley (CA); Alexei J. MARKO, Vancouver (CA)

(21) Appl. No.: 15/244,312

(22) Filed: Aug. 23, 2016

Related U.S. Application Data
(60) Provisional application No. 62/209,815, filed on Aug. 25, 2015.

(51) Int. Cl. A61B 5/00; A61B 17/435; A61M 31/00; A61B 10/02; A61B 8/00

(52) U.S. Cl. CPC A61B 5/4839; A61B 10/0241; A61B 8/4455; A61M 31/00; A61B 10/0291; A61B 17/435; A61B 2017/3413

(57) ABSTRACT

A biopsy needle sterilant device includes a sterilant pouch holding a sterilant and a sterilant actuation mechanism in fluid communication with the sterilant pouch. Actuation of the sterilant actuation mechanism delivers the sterilant from the sterilant pouch toward a target tissue. Actuation may be performed manually without requiring an external power source. The sterilant device may be coupled to a biopsy needle guide or an ultrasound probe.

### CROSS-REFERENCE

Present application claims benefit of U.S. Provisional Application No. 62/209,815 (Attorney Docket No. 49045-703.101) filed Aug. 25, 2015; the entire contents of which are incorporated herein by reference.

### BACKGROUND OF THE INVENTION

1. Field of the Invention

The present invention is related to medical devices, systems, and methods. More particularly, the present invention is related to biopsy needles.

Biopsy needles are commonly used to obtain tissue samples for diagnostic purposes. In the case of a skin biopsy, the external surface of the skin may be easily wiped and cleaned with an agent such as a sterilant, or an antiseptic or disinfectant before performing the procedure thereby minimizing the possibility of contamination and infection. However, in some cases performing a biopsy may require the biopsy needle to pass through unsanitized regions of the body which may contaminate the tissue sample and may cause infection of the target tissue at the biopsy site. For example, biopsies are often performed in patients in whom prostate cancer may be suspected due to an enlarged prostate or another screening test such as the prostate specific antigen (PSA) test. The standard biopsy procedure typically involves inserting the biopsy needle into the patient's rectum and passing the biopsy needle through the rectal mucosal tissue layer into the prostate. It may be difficult to clean and disinfect the rectal mucosal tissue layer and therefore the biopsy needle may become contaminated and this contamination may be passed to the site of the needle biopsy potentially resulting in infection.

Others have proposed biopsy needles with various attachments (e.g. a foam barrier soaked with sterilant) through which the needle passes in order to sanitize the needle, yet these devices do not always effectively sanitize the contaminated tissue or may be expensive or difficult to operate. Some of these devices do not cooperate easily with other equipment used during a biopsy procedure, such as an ultrasound probe which may be used to help guide the biopsy needle to the target tissue.

It would therefore be desirable to overcome at least some of these challenges and provide a biopsy needle that can clean or sanitize tissue or other material that the needle must pass through on its way to the target tissue. It would be desirable if such a biopsy needle or system for cleaning or sanitizing is also compatible with other equipment that may be used during the procedure, such as an ultrasound probe. It would further be desirable if the biopsy needle were inexpensive and easy to use. At least some of these objectives will be satisfied by the devices described herein.

2. Description of the Background Art

Biopsy needles are well known in the art, including the following U.S. Pat. Nos. 6,575,992; 6,447,482; 6,171,293; 5,733,252; 5,398,690; 5,392,766; 5,092,845; 5,015,228; 4,989,614; 4,874,364; 4,517,702; 4,507,118; 4,351,616; 3,587,575; 3,354,881; 2,888,924; and 1,921,034.

### SUMMARY OF THE INVENTION

The present invention is related to medical devices, systems, and methods. More particularly, the present invention is related to biopsy needles.

The term “sterilant” is used herein to indicate an agent that either sanitizes, disinfects, or otherwise controls, reduces, or eliminates microbial contamination. Such a sterilant includes but is not limited to a disinfectant, an antiseptic, or an antibiotic. Similarly, “sterilizing” as used herein refers to sanitizing, disinfecting, or otherwise controlling, reducing, or eliminating microbial contamination and may be done by any sterilant described herein.

In a first aspect of the present invention, a biopsy needle sterilant device comprises a sterilant pouch holding a sterilant therein, and a sterilant actuation mechanism in fluid communication with the sterilant pouch. Actuation of the sterilant actuation mechanism delivers the sterilant from the sterilant pouch toward a target tissue. Optionally, actuation of the actuation mechanism may be done passively, wherein actuation is done manually without requiring an external power source.

The device may further comprise a coupling element for releasably coupling the device with an ultrasound probe. The device may also further comprise a needle guide having a needle lumen extending therethrough and configured to receive and guide a biopsy needle to target tissue. The device may include a biopsy needle disposed in the needle lumen. The sterilant may contact the biopsy needle. The needle guide may have a sterilant lumen extending therethrough and the sterilant lumen may be configured to deliver the sterilant from the sterilant pouch toward the target tissue. The sterilant lumen may also comprise a nozzle interface that increases sterilant velocity exiting the sterilant lumen, preferably dispersing sterilant between the lumen outlet and the contacting tissue. Furthermore, the device may comprise a sensor-based detection system to detect proximity of a portion of the device, such as a distal end, to a target tissue and that automatically controls dispersion of the sterilant.

In another aspect of the present invention, a biopsy needle system comprises a biopsy needle guide device and a biopsy needle slidaby disposed in the biopsy needle guide device. The device also comprises a sterilant delivery system holding a sterilant therein and having an actuation mechanism. Actuation of the actuation mechanism delivers the sterilant to target tissue and to the biopsy needle.

The system may further comprise an ultrasound probe releasably coupled to the biopsy needle guide device. Actuation of the actuation mechanism may be performed passively, wherein actuation is done manually without requiring power from an external power source.

In still another aspect of present invention, a method for performing a biopsy comprises positioning a biopsy needle into a body region, such as a body cavity, and adjacent target tissue, applying a sterilant onto a layer of tissue disposed between the biopsy needle and the target tissue, and applying the sterilant onto the biopsy needle. The method also comprises penetrating the needle through the layer of tissue, and biopsying the target tissue.

The method may further comprise introducing sterilant to the puncture sight for a specified period of time in order for sterilizing action to occur prior to puncturing tissue.

Applying the sterilant onto the layer of tissue or onto the biopsy needle may comprise automatically actuating an actuation mechanism to deliver sterilant from a sterilant pouch to the layer of tissue or onto the biopsy needle. The power required to actuate the actuation mechanism may come from an external power source, or actuation may be performed passively without applying external power, such as actuation by an operator's hand squeezing or otherwise actuating the actuation mechanism. Automatically actuating the actuation mechanism to deliver sterilant from the sterilant pouch to the layer of tissue or onto the biopsy needle may comprise detecting the proximity of a portion of the device, such as a distal end, to a target tissue.

The body cavity may be a rectum and the target tissue may be a prostate. The body cavity may be a vagina and the target tissue may be an ovary or a fallopian tube. The layer of tissue may be a layer of mucosal tissue.

Positioning the biopsy needle may comprise guiding the biopsy needle with an ultrasound probe. Guiding the biopsy needle may comprise advancing the biopsy needle through a lumen in a biopsy needle guide.

Applying the sterilant onto the layer of tissue or onto the biopsy needle may comprise manually actuating an actuation mechanism to deliver the sterilant from a sterilant pouch to the layer of tissue or onto the biopsy needle. Application of the sterilant may comprise spraying, misting, dripping, or flooding a target area with the sterilant. Power from an external power source may not be required to actuate the actuation mechanism.

Biopsying the target tissue may comprise biopsying a prostate or a portion of the female reproductive system. Biopsying the target tissue may comprise harvesting eggs from an ovary or fallopian tube.

The method may further comprise coupling a biopsy needle guide with an ultrasound probe.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic diagram of a needle biopsy procedure.

FIG. 2 is a perspective view of an exemplary embodiment of the device coupled to an ultrasound probe.

FIG. 3 is a perspective view of the device in FIG. 2 without the ultrasound probe.

FIG. 4 is a cross-sectional view of the device in FIG. 3.

FIG. 5 is an exploded-view of the device and ultrasound probe in FIG. 2.

FIGS. 6A-6E illustrate use of the device in FIGS. 1-5 to perform a biopsy.

FIG. 7 illustrates a side view of the device in FIG. 2 with a porous interface coupled to its distal end.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

While exemplary embodiments will be primarily directed at biopsy needles for use with the prostate, one of skill in the art will appreciate that this is not intended to be limiting, and the devices described herein may be used for other therapeutic or diagnostic procedures and in other anatomical regions of a patient's body.

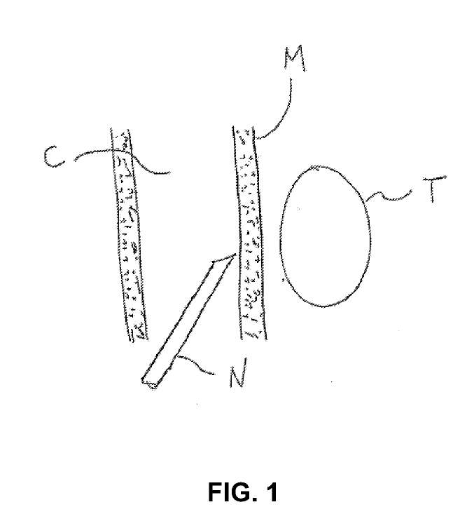

FIG. 1 shows an optional needle biopsy procedure where the biopsy needle N is inserted into a body cavity C. The biopsy needle N has to pass through mucosal tissue M or another layer of tissue in order to reach the target tissue T. The mucosal layer of tissue may be contaminated and therefore the needle passing therethrough may also become contaminated. For example, the body cavity C may be a patient's rectum and the target tissue T may be the prostate. The mucosal layer of tissue may be contaminated with bacteria or fecal matter. In another option, the body cavity may be a patient's vagina and the target tissue may be an ovary or fallopian tube during egg harvesting or in-vivo fertilization procedures. In other options, the needle may be passed through any barrier tissue in order to reach target tissue, such as in a nasal, oral, gastrointestinal, urinary tract, uterine or any other passageway. Therefore, the devices and methods described herein may apply to any procedure where an instrument such as a needle has to pass through tissue in order to reach target tissue and wherein it may be advantageous to sanitize, clean, or otherwise sterilize the tissue to avoid or minimize contamination of the needle and the target tissue.

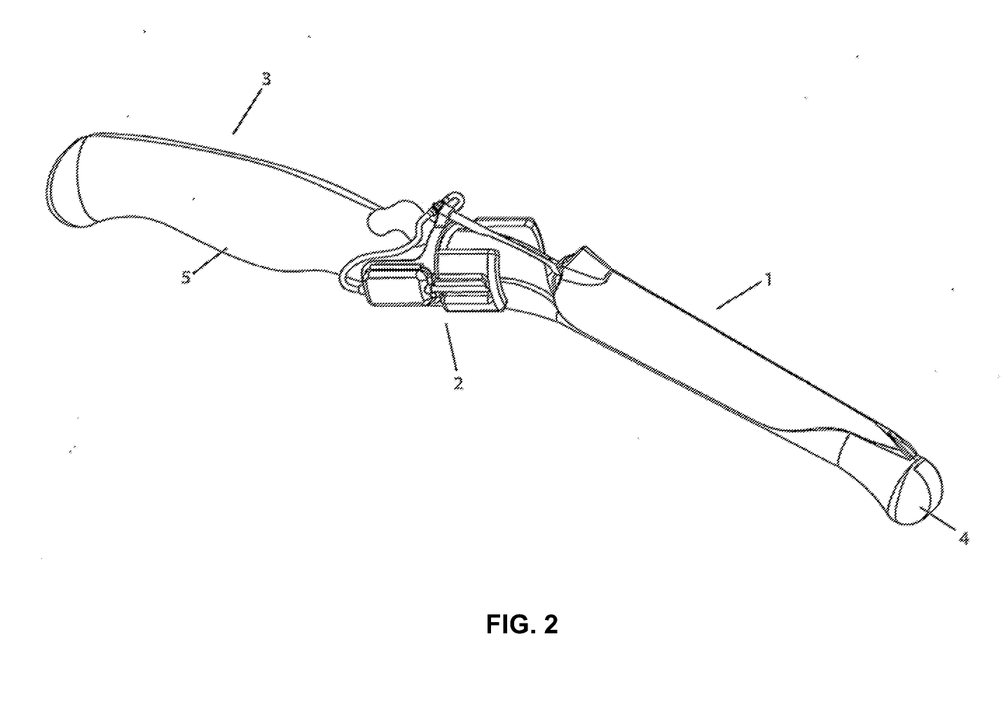

FIG. 2 illustrates an exemplary device having a needle guide 1 which is coupled to a sterilant delivery system 2. The needle guide 1 is preferably designed with geometries that enable the device to be coupled to an ultrasound probe 3, in close proximity to a sensor 4 that is located distally to the handle 5 of the ultrasound probe 3. The sterilant delivery system 2 is also designed with geometries that enable the device to be coupled to an ultrasound probe 3 and/or to the needle guide 1 in a location that is optimal to the operator, allowing for the controlled delivery of a sterilant, which may comprise a fluid, a mist, a powder, a gel, or any combination thereof.

The sensor 4, as it is described herein, may refer to one or more sensor types used to sense a property or characteristic of the device, system, method of delivery, method of treatment, and/or target sites, including but not limited to any human tissue and those described previously. The sensor 4 may be a proximity sensor to detect the proximity of a target site, such as a mucosal tissue M or a target tissue T, to some portion of the device, such as a distal end. Possible proximity sensors include but are not limited to a capacitive sensor, a capacitive displacement sensor, a Doppler effect sensor, an Eddy-current sensor, a fiber optic sensor, a Hall effect sensor, an inductive sensor, an infrared sensor, a laser rangefinder, a magnetic sensor, an optical sensor (including passive optical sensor, such as charge-coupled devices), a passive thermal infrared sensor, a photocell, radar, sonar, or an ultrasonic sensor, or any combination thereof. The sensor 4 may be used in combination with a controller (not illustrated) to: regulate the delivery of sterilant to a target site based on the proximity of some portion of the device to the target site automatically, the regulation including altering the application area of the sterilant, the velocity of the sterilant, and/or whether or not sterilant is delivered; and/or regulate the delivery of the needle N to a target site based on the proximity of some portion of the device to the target site, including controlling whether the needle N is delivered to a target site, penetrates or punctures a mucosal layer, penetrates or punctures a target tissue, the speed at which the needle N is delivered, and/or the depth to which the needle travels. The sensor 4 may be configured to take data collected by the ultrasound probe 3, such as the depth of the target site, the size, shape, and position of the target site, the position of the needle, the position of the needle with respect to the target site, and/or the position of the target site with respect to the needle, and transmit that data to the controller to control the operation of the device including all the ways described herein.

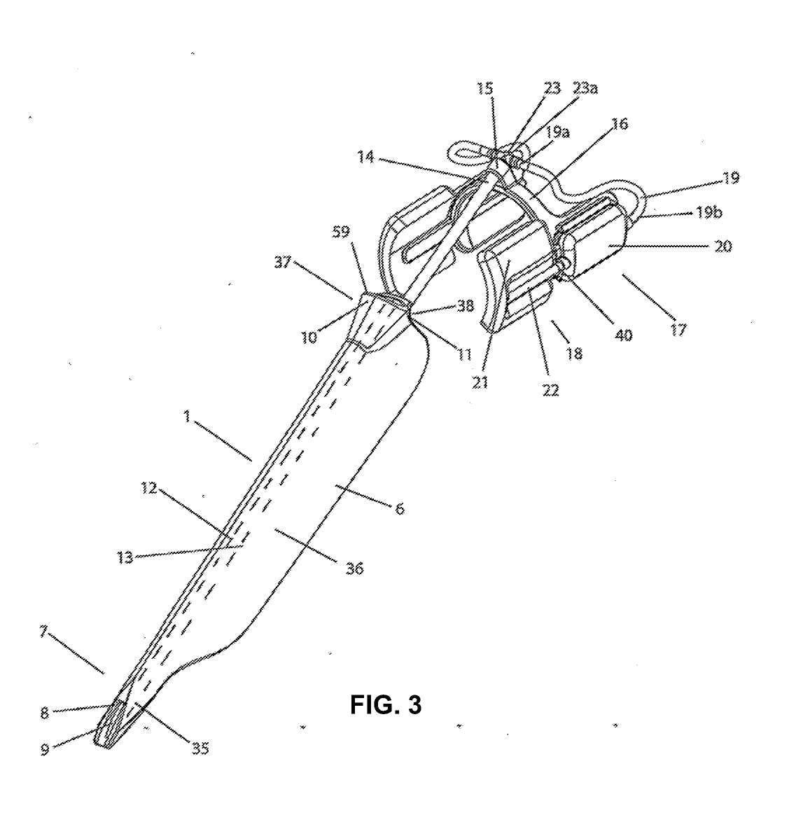

Referring now to FIG. 3, the needle guide 1 comprises a narrow, rigid, and streamlined element 36, a distal tip section 7, a middle section 6, and a proximal section 37. The middle section 6 enables the needle guide 1 to be releasably coupled to an ultrasound probe 3. This may be by clipping or otherwise coupling the two together. The element 36 may have a concave inner surface which conforms to the contours of the ultrasound probe in order to allow the two components to snap, clip, or otherwise be releasably or fixedly coupled together, and the outer surface may be convex, thereby providing a low profile device which can easily be inserted into a body cavity. The distal tip section 7 of the needle guide 1 includes a nozzle 35, with two inner lumens that may be stacked vertically on top of each other (12, 13, as shown), disposed with one in the other, such as the needle lumen within the sterilant lumen, or they may be side by side. The needle lumen 12 has a distal needle outlet 8, and a proximal needle inlet 10. A biopsy needle (not shown) may be inserted into the needle guide 1 at the proximal needle inlet 10, and through the needle lumen 12, whereupon it will protrude from the distal needle outlet 8. Additionally, the sterilant lumen 13 has a distal sterilant outlet 9, and a proximal sterilant inlet 11. The nozzle 35 may be such that: it may increase the velocity (either exiting the nozzle or passing through a lumen) of a fluid, such as sterilant, passing through the needle lumen 12 or the sterilant lumen 13, or both; it may broaden the spread of the fluid that passes through and exits the nozzle 35 onto a target site; it may narrow the spread of the fluid that passes through the and exits nozzle 35 onto a target site; and/or if the sterilant is a misted or powdered form it may control the size, dimensions, shape, and/or distribution of particles of the sterilant as they pass through and exit the nozzle 35 to a target site in order to optimize coating of the target site in the fluid, preferably sterilant.

The proximal section 37 of the needle guide 1 is comprised of the proximal needle inlet 10, the proximal sterilant inlet 11, a conical needle director 59, and an attachment point 38 for a coupling 14 which connects the needle guide 1 to the sterilant delivery system 2. The conical needle director has an aperture for receiving a needle and an inner conically tapered surface that allows the needle to be easily aligned with and loaded into the needle inlet without getting hung up on edges or other protrusions which would prevent advancement of the needle.

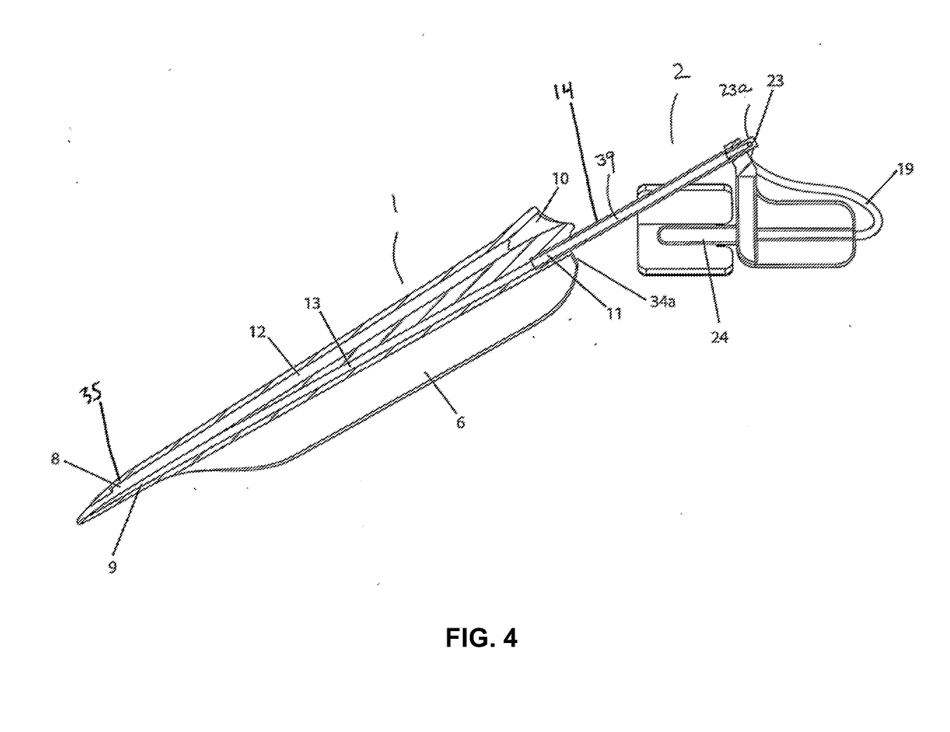

The hollow coupling 14 is comprised of a coupling lumen 39 (best seen in FIG. 4) which allows for the transport of sterilant from the sterilant delivery system 2 into the proximal sterilant inlet 11, and is preferably stiff and tubular. The attachment point 38 comprises the distal section of the coupling 14, while a carriage attachment point 15 and t-joint 23 comprises the proximal section of the coupling 14. The carriage attachment point 15 serves as the junction between the coupling 14, and the carriage 16.

The carriage 16—a structural element meant to hold several elements described herein in place—is preferably symmetric and allows users of both left and right handedness to operate the device by providing access to a balloon trigger 20 on either side of the device. Each side of the carriage 16 is coupled with a sterilant tube 19, a balloon trigger 20, and a sterilant pouch 21, and maintains features which enable the attachment of the features to the carriage 16. These features will be further described below.

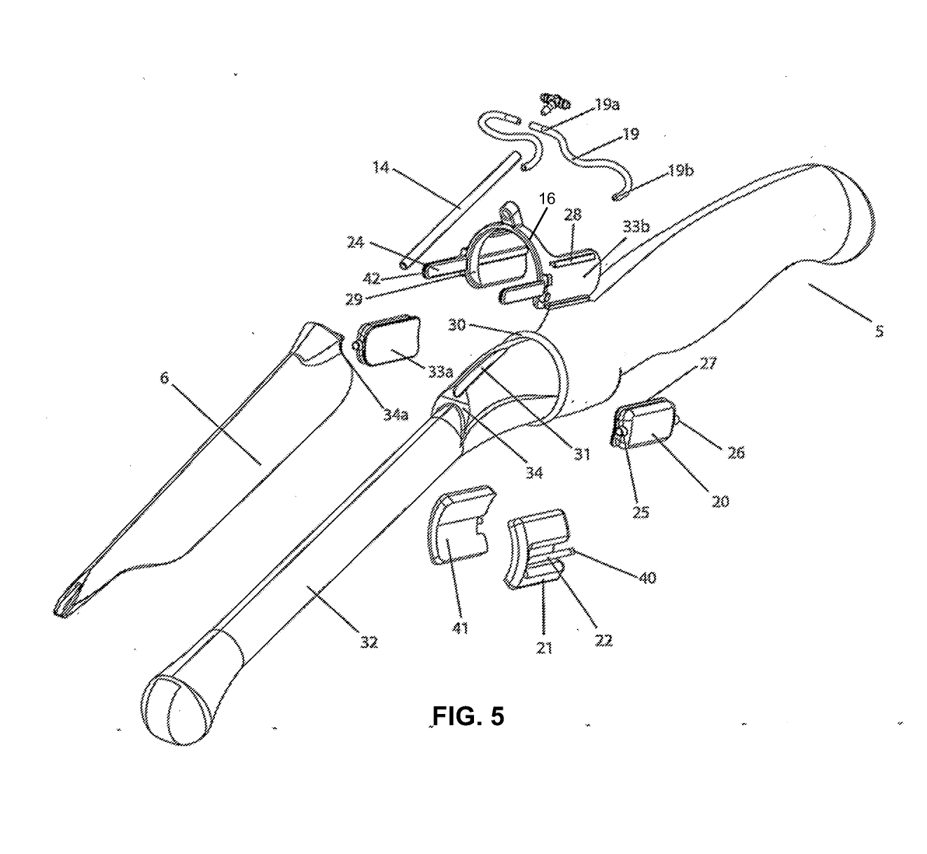

The balloon trigger 20 may be a mechanism having a resilient or deformable region that may be manually depressed inward and that is biased to return to the unbiased outward position. When an operator actuates the balloon trigger by depressing and releasing the balloon trigger 20, a vacuum is created within the balloon trigger 20 due to the presence of a first one-way valve 40 and a second one-way valve 23a. The first one-way valve 40 (best seen in FIG. 5) prevents a positive pressure gradient from forming in the direction from balloon trigger 20 to transfer tube 22, and the second one-way valve 23a prevents a positive pressure gradient from forming in the direction from tubing outlet 19a to tubing inlet 19b. The vacuum created within the balloon trigger 20, coupled with the plurality of one-way valves 40 and 23a transports sterilant from within the sterilant pouch 21 into the balloon trigger. Once the balloon trigger 20 is filled with sterilant, additional actuation by pressing the trigger will cause sterilant to be transported from the balloon trigger 20 to the tubing inlet 19b, through the tubing 19 to the tubing outlet 19a, into the t-joint 23, down the coupling lumen 39, down the sterilant lumen 13 and out of the nozzle 35 at the distal sterilant outlet 9. Once at the nozzle 35, the sterilant may saturate both the tip of the biopsy needle (not shown) and the site of puncture. The sterilant may be applied either directly or indirectly to the needle body, the needle tip, the barrier tissue, the target tissue, or any combination thereof.

FIG. 4 is a cross-sectional view of the needle guide 1 and sterilant delivery system 2 that focuses on the internal path through which sterilant flows under operation. It should be readily apparent that once the sterilant has been actuated from the sterilant pouch to the balloon trigger through an initial priming, through actuation of the balloon trigger, or by having been disposed originally in the balloon trigger, the sterilant will be transferred from the sterilant tube 19, through the t-joint 23 coupled to a second one-way valve 23a, through a coupling lumen 39 of the coupling 14, through the sterilant lumen 13 and out of the nozzle 35 at the junction of the distal sterilant outlet 9 and distal needle outlet 8. The first one-way valve (best seen in FIG. 5) and second one-way valve 23a may cause the device to bias the flow of sterilant along a single path or direction, such as from the sterilant pouch through tubing to the sterilant lumen and then onto a target site. The first one-way valve and second one-way valve 23a may preferentially cause sterilant to flow out of the device. In alternative embodiments the first one-way valve and the second one-way valve 23a may not cause the device to bias flow of sterilant along a single path or direction. The sterilant may flow from or to the sterilant pouch through the balloon trigger to or from the sterilant lumen.

Referring now to FIG. 5, shown is an exploded view of the device described above coupled with an exemplary ultrasound probe. One of skill in the art will appreciate that this is not intended to be limiting and that the sterilant device is optionally coupled to an ultrasound probe or may remain uncoupled to the ultrasound probe. Moreover, the ultrasound probe described herein is merely one exemplary ultrasound probe and it is not intended to be limiting. The sterilant device may optionally be coupled to any ultrasound probe or other guidance device. The features described herein with respect to the ultrasound probe are optional, and other ultrasound probes may have some or all these features. Turning specifically to the methods through which certain features of the invention are coupled to each other, we see that there are features of the generic ultrasound probe 5 that can be used to couple the present invention securely and accurately prior to use by a physician or operator. In the mid-section of the ultrasound probe 5 there is a cylindrical slot 31 which acts as a locating feature and maintains the location of the coupling 14 of the sterilant delivery system 2. The cylindrical slot 31 also prevents the rotation of the sterilant delivery system 2 with respect to the needle guide 1. Additionally, there is a shoulder feature 34 present on the ultrasound probe 5 which also acts to aid in the location of the needle guide 1 by providing a surface upon which the needle guide proximal end 34a can rest. Finally, there is a circular ring 30 present in the mid-section of the ultrasound probe 5 that also acts as a shoulder and that provides a surface upon which the clipping surface 29 of the carriage 16 may abut.

The slender and elliptical main shaft 32 of the ultrasound probe 5 is typically shaped in such a manner as to provide a location for the middle section 6 of the needle guide 1 to attach by pressing the needle guide onto the elliptical main shaft 32 until it clips into place either releasably or fixedly.

Turning now to the carriage 16, several features enabling component attachment will be detailed. On the sterilant pouch 21 there is a first one-way valve 40 which is further connected to the transfer tube 22 of the sterilant pouch 21. Sterilant passes through one-way valve 40 and is transferred to inlet port 25 on the balloon trigger 20. The sterilant pouch 21 may comprise a thin section 41 that allows for connection between the carriage 16 and the sterilant pouch 21 by means of a clipping arm 24 that has a thin slit 42 which mates with and clasps onto the thin section 41. The clipping arm 24 also supports and suspends the sterilant pouch 21 away from the surface of the ultrasound probe 5.

The balloon trigger 20 provides location for a sterilant outlet port 26 that is in connection with a tubing inlet 19b. The sterilant inlet port 25 and outlet port 26 act together to transfer sterilant from the sterilant pouch 21 through the balloon trigger 20 and into the tubing 19. Also seen on the balloon trigger 20 is a perimeter flange 27 that provides purchase for the carrier slots 28 to grasp the balloon trigger 20. The balloon trigger 20 has a curved surface 33a that abuts against a similar curved surface 33b on the sterilant carrier 16.

This present disclosure preferably relates to a hand-held biopsy needle guiding device that enables the administration of sterilants directly to the tip of the biopsy needle, and to the site of the biopsy needle puncture in the rectum or vagina of a patient, and significantly reduces the spread of infection into and around the lesion left after a tissue core sample is removed from the bacteria rich environment, as well as minimizing or preventing contamination of the tissue core sample obtained by the biopsy needle.

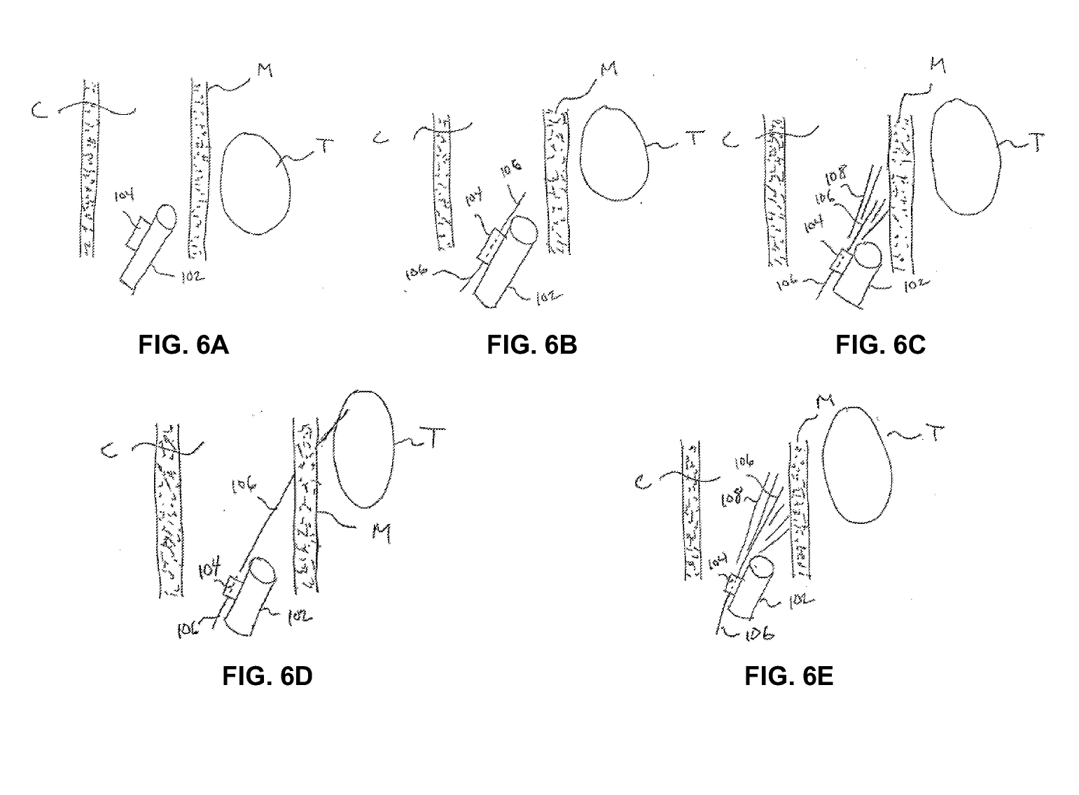

FIGS. 6A-6E illustrate an exemplary method of using any of the devices described herein. In FIG. 6A, a probe 102 such as an ultrasound probe is used to guide the biopsy needle guide device 104 with sterilant delivery through the body cavity C to a position adjacent the target treatment tissue T. The biopsy needle guide device 104 may also be any of the guide devices with sterilant delivery described herein. A layer of mucosal tissue M forms a barrier between the probe 102 and the target tissue. In this example, the cavity C may be a patient's rectum and the target tissue is the prostate. The barrier tissue is a layer of mucosal tissue between the rectum and the prostate. In another example, the body cavity may be the vagina and the target tissue is an ovary or a fallopian tube.

Once the needle guide has been properly positioned adjacent the target tissue, the biopsy needle 106 may be advanced through the needle guide toward the target tissue, as seen in FIG. 6B.

In FIG. 6C, the sterilant delivery mechanism on the needle guide 104 is actuated so that sterilant 108 is sprayed distally from the needle guide device to sanitize the needle 106 and the mucosal layer of tissue M. The sterilant delivery mechanism may be any of those described herein. In this example, actuation is by depressing the balloon trigger by hand, and therefore an external power source is not required to pump and deliver the sterilant. This may be described as a passive actuation system. Optionally, in any of the embodiments disclosed herein, a powered pump or other actuation device requiring external power may be used to actuate the balloon trigger or directly pump the sterilant from the sterilant pouch, out the needle guide and to the target tissue.

In FIG. 6D the biopsy needle is advanced further distally to pass through the layer of mucosal tissue and into the target tissue. In the case where the target tissue is a prostate, the prostate is then biopsied, or in the case of an ovary or fallopian tube, the biopsy is performed or the eggs are harvested. Or in other variations, in vivo fertilization may be performed.

In FIG. 6E, the needle is withdrawn proximally from the target tissue and from the mucosal layer of tissue. Optionally, another actuation of the sterilant delivery mechanism sprays additional sterilant onto the needle and mucosal tissue layer further disinfecting the needle and tissue before the device is removed from the cavity C.

For any of the embodiments described herein, but especially those of FIGS. 6A-6E, any procedure may comprise allowing a user to deliver sterilant to a target region (such as a mucosal layer of tissue M or a target treatment tissue T), waiting some period of time so that the sterilant may act (such as to kill bacteria or other microbes or reduce their count), then continuing the procedure. The period of time may be at the user's preference, a function of the rate at which bacteria or other microbes are killed, a function of the rate at which sterilant is absorbed, evaporated, or converted into a non-usable form, or a pre-ordained amount that can range anywhere from about one to three seconds, about one to ten seconds, about ten to thirty seconds, or about one second to sixty seconds. Given the myriad ways in which a user may wish to utilize a pause in a procedure, a complete listing is omitted. However, one of skill in the art will appreciate the factors relating to utilizing a pause after delivering a sterilant. Such a pause may also be iteratively coupled to any relevant step such that the pause and the relevant step are repeated any number of times. One may envision, for example, a procedure wherein a biopsy needle is positioned into a body cavity and adjacent a target tissue, applying a sterilant onto a layer of tissue disposed between the biopsy needle and the target tissue, waiting some period of time so that the sterilant may act, applying the sterilant again to the layer of tissue disposed between the biopsy needle and the target tissue, waiting some period of time so that the sterilant may act, applying the sterilant onto the biopsy needle, penetrating the needle through the layer of tissue, and biopsying the target tissue. The above example is intended to illustrate the concept and should not be interpreted as a requirement or necessary.

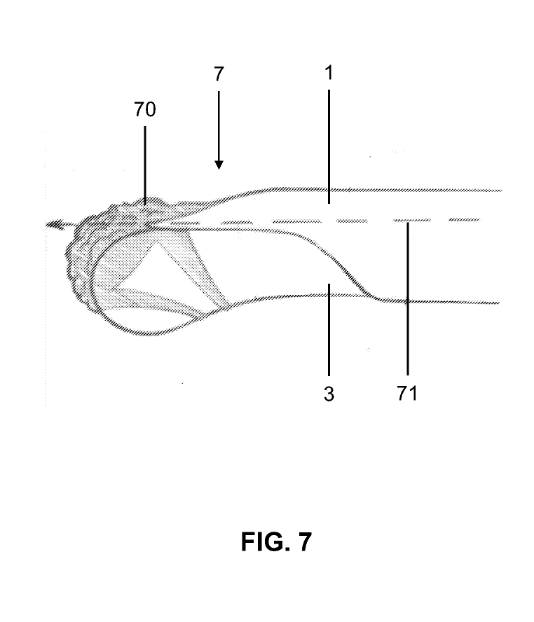

FIG. 7 shows a side view of the device in FIG. 2 with an optional porous interface 70 coupled to its distal end. In the illustrated embodiment, the porous interface 70 is physically coupled to a distal end of an ultrasound probe 3 and contacts the distal tip section 7 of the needle guide 1. The porous interface 70 may be comprise a sponge or a sponge-like pad and may aid in swabbing or flooding a tissue or region with sterilant. In some embodiments, the porous interface 70 may contact a tissue or region directly, while in other embodiments the porous interface 70 may not directly contact a tissue or region. In the illustrated example, the axis defining the needle path 71 passes through a portion of the porous interface 70. In other embodiments the axis defining the needle path 71 may pass through the center of the porous interface, pass through a portion of the porous interface 70, pass tangentially on some edge of the porous interface 70, or it may not pass through any portion of the porous interface. In other embodiments, the porous material 70 may couple to the ultrasound probe 3 or the distal tip section 7 of the needle guide 1, or both.

For any of the embodiments herein described, a sensor (as illustrated in FIG. 2) may be used to automatically actuate an actuation mechanism to deliver sterilant from a sterilant pouch to a layer of tissue or onto a biopsy needle. The power required to actuate the actuation mechanisms for such embodiments may be either an internal power source of the device or an external power source. Such automatic actuation of an actuation mechanism to deliver sterilant from a sterilant pouch to a layer of tissue or onto the biopsy needle may comprise a sensor capable of detecting proximity of a target site to some portion of the device.

In any of these devices, a sterilant, antiseptic, disinfectant, or other agent used to kill bacteria and other microbes may be used. Therefore any agent that reduces the possibility of contamination or infection may be used. Other therapeutic agents may also be delivered alone or in combination with the sterilant, and thus the system is not limited to a sterilant delivery system but may also be a delivery system for any therapeutic agent. For example, antibiotics may be delivered, or vasodilators or constrictors may be delivered to control localized bleeding. Dyes or other indicators may be delivered to help visualize the target tissue when illuminated with specific wavelengths of light. The system may also be used to irrigate the target tissue with saline or other fluids.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A biopsy needle sterilant delivery device, said device comprising:
a sterilant pouch holding a sterilant therein;
a sterilant actuation mechanism in fluid communication with the sterilant pouch, wherein actuation of the sterilant actuation mechanism delivers the sterilant from the sterilant pouch toward a target tissue.

2. The device of claim 1, wherein actuation of the actuation mechanism is performed manually without requiring an external power source.

3. The device of claim 1, further comprising a coupling element for releasably coupling the device with an ultrasound probe.

4. The device of claim 1, further comprising a needle guide having a needle lumen extending therethrough and configured to receive and guide a biopsy needle.

5. The device of claim 4, further comprising a biopsy needle disposed in the needle lumen.

6. The device of claim 1, further comprising a needle guide having a sterilant lumen extending therethrough and configured to deliver the sterilant from the sterilant pouch toward the target tissue.

7. The device of claim 6, wherein the sterilant is discharged from the sterilant lumen and coats the needle.

8. The device of claim 7, further comprising a needle guide having a sterilant lumen with a porous interface for preferably dispersing sterilant between the lumen outlet, and the contacting tissue.

9. The device of claim 7, further comprising a needle guide having a sterilant lumen with a nozzle interface that increases fluid velocity and preferably disperses sterilant between the lumen outlet and the contacting tissue.

10. The device of claim 7, further comprising a needle guide having a sensor-based detection system for detecting proximity to target tissue and then automatically dispersing sterilant.

11. A biopsy needle system, said system comprising:
a biopsy needle guide device;
a biopsy needle disposed in the biopsy needle guide device; and
a sterilant delivery system holding a sterilant therein and having an actuation mechanism, wherein actuation of the actuation mechanism delivers the sterilant to target tissue and to the biopsy needle.

12. The system of claim 11, further comprising an ultrasound probe releasably coupled to the biopsy needle guide device.

13. The system of claim 11, wherein actuation of the actuation mechanism is performed manually without requiring power from an external power source.

14. A method for performing a biopsy, said method comprising:
positioning a biopsy needle into a body cavity and adjacent target tissue;
applying a sterilant onto a layer of tissue disposed between the biopsy needle and the target tissue;
applying the sterilant onto the biopsy needle;
penetrating the needle through the layer of tissue; and
biopsying the target tissue.

15. The method of claim 14, further comprising introducing to a puncture sight for a specified period of time in order for sterilizing action to take place prior to puncturing tissue.

16. The method of claim 14, wherein applying the sterilant onto the layer of tissue or onto the biopsy needle comprises automatically actuating an actuation mechanism to deliver sterilant from a sterilant pouch to the layer of tissue or onto the biopsy needle.

17. The method of claim 16, wherein automatically actuating an actuation mechanism to deliver sterilant from a sterilant pouch to the layer of tissue or onto the biopsy needle comprises providing power from an external power source to the actuation mechanism.

18. The method of claim 16, wherein automatically actuating an actuation mechanism to deliver sterilant from a sterilant pouch to the layer of tissue or onto the biopsy needle comprises detecting tissue proximity with a sensor.

19. The method of claim 14, wherein the body cavity is a rectum and the target tissue is a prostate.

20. The method of claim 14, wherein the body cavity is a vagina and the target tissue is an ovary or a fallopian tube.

21. The method of claim 14, wherein the layer of tissue is a layer of mucosal tissue.

22. The method of claim 14, wherein positioning the biopsy needle comprises guiding the biopsy needle with an ultrasound probe.

23. The method of claim 22, wherein guiding the biopsy needle comprises advancing the biopsy needle through a lumen in a biopsy needle guide.

24. The method of claim 14, wherein applying the sterilant onto the layer of tissue or onto the biopsy needle comprises manually actuating an actuation mechanism to deliver the sterilant from a sterilant pouch to the layer of tissue or onto the biopsy needle, and wherein power is not required from an external power source to actuate the actuation mechanism.

25. The method of claim 14, wherein applying comprises spraying, misting, dripping, or flooding the region with sterilant.

26. The method of claim 14, wherein biopsying the target tissue comprises biopsying a prostate.

27. The method of claim 14, wherein biopsying the target tissue comprises biopsying a portion of the female reproductive system.

28. The method of claim 14, wherein biopsying the target tissue comprises harvesting eggs from an ovary or fallopian tube.

29. The method of claim 14, wherein biopsying the target tissue comprises conducting an in vivo fertilization.

30. The method of claim 14, further comprising coupling a biopsy needle guide with an ultrasound probe.
